(19) United States
(12) Patent Application Publication
(10) Pub. No.: US 2022/0172829 A1
(43) Pub. Date: Jun. 2, 2022

(54) Techniques For Delivering Real-Time Point Of Care Messaging To Health Care Providers

(71) Applicant: OPTIMIZERx Corporation, Rochester, MI (US)

(72) Inventors: Adam ALMOZLINO, Newton, MA (US); Stephen SILVESTRO, Hollis, NH (US)

(73) Assignee: OPTIMIZERx Corporation, Rochester, MI (US)

(21) Appl. No.: 17/189,820

(22) Filed: Mar. 2, 2021

Related U.S. Application Data
(60) Provisional application No. 63/119,487, filed on Nov. 30, 2020.

(51) Int. Cl. G16H 40/20; G16H 10/60; G16H 50/20; G16H 20/10; G16H 10/20; G16H 10/40; G16H 70/20; G16H 70/40; G06N 3/08

(52) U.S. Cl. CPC G16H 40/20; G16H 10/60; G16H 50/20; G16H 20/10; G06N 3/08; G16H 10/40; G16H 70/20; G16H 70/40; G16H 10/20

(57) ABSTRACT

Techniques are presented for delivering point of care message content to a healthcare provider. Upon receiving input data from a given healthcare provider, a determination is made regarding an aspect of therapy or an indicator thereof for the given patient using machine learning, where the determination is based in part on the input data. The input data pertains to a particular encounter between a given patient and a healthcare provider. Particular message content for the healthcare provider is then determined using the predicted aspect of therapy or the indicator thereof for the given patient and criteria for delivering message content to a healthcare provider. The particular message content is preferably related to a service or product associated with a therapy for the given patient. Finally, the particular message content is delivered to the healthcare provider during the encounter with the given patient.

### CROSS-REFERENCE TO RELATED APPLICATIONS

This application claims the benefit of U.S. Provisional Application No. 63/119,487, filed on Nov. 30, 2020. The entire disclosure of the above application is incorporated herein by reference.

### FIELD

The present disclosure relates to improved techniques for delivering real-time point of care messaging to healthcare providers.

### BACKGROUND

Recent developments in pharmaceuticals and healthcare delivery have led to the development of medications and therapies that can treat or alleviate many medical conditions or diseases that were incurable in the past. Some pharmaceutical companies that manufacture medications expend vast resources towards research and development. Most of the cost associated with extensive research and development is passed on to patients in the form of higher prices for medications.

Furthermore, due to the sheer number of possible disease states a patient may experience and medical treatments applicable for those diseases, it is difficult for healthcare providers to discover medications that might be most effective in disease treatment. It is desirable to present healthcare providers with information regarding clinical conditions, medications, and/or medical treatments during a patient encounter with the healthcare provider. More specifically, it is desirable to improve the point of care messaging delivered to the healthcare providers during a patient encounter.

This section provides background information related to the present disclosure which is not necessarily prior art.

### DRAWINGS

The drawings described herein are for illustrative purposes only of selected embodiments and not all possible implementations, and are not intended to limit the scope of the present disclosure.

FIG. 1 is a diagram of an example system for delivering point of care message content to a healthcare provider.

FIG. 2 is a flowchart showing an improved technique for delivering point or care message content to a healthcare provider.

FIG. 3 is a flowchart showing an example implementation for delivering point of care message content to a healthcare provider.

FIG. 4 is a diagram depicting an example embodiment for the messaging provider application.

Corresponding reference numerals indicate corresponding parts throughout the several views of the drawings.

### DETAILED DESCRIPTION

Example embodiments will now be described more fully with reference to the accompanying drawings.

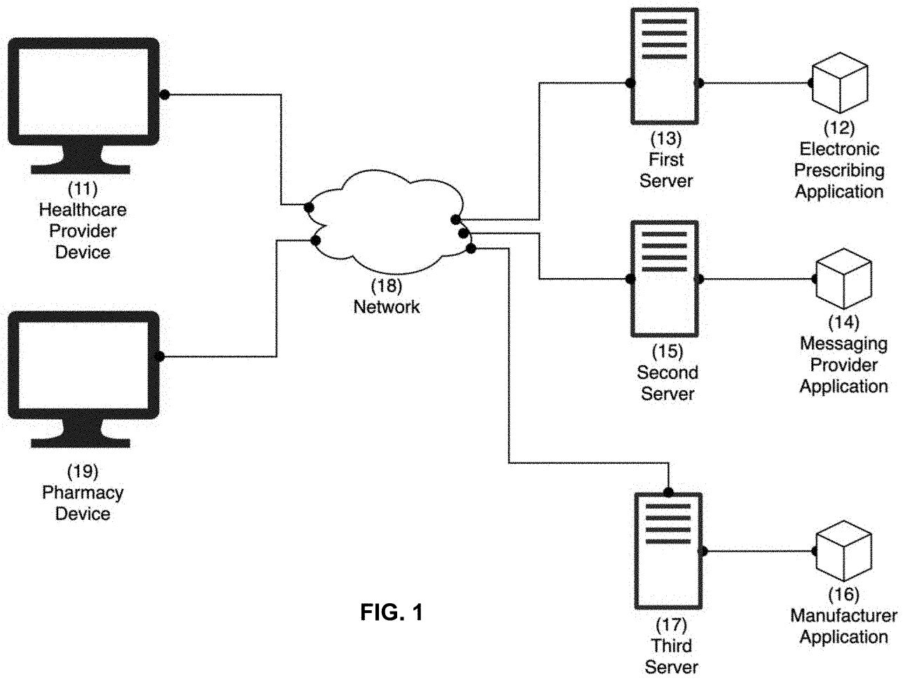

FIG. 1 illustrates an example system 10 for delivering point of care message content to a healthcare provider. The system 101 is comprised generally of a healthcare provider device 11, an electronic prescribing application 12 that is accessible to healthcare providers through the healthcare provider device 11, and a messaging provider application 14 that provides message content to healthcare providers via the electronic prescribing application 13. The system 10 may optionally include a pharmacy device 19 that electronically receives prescriptions for medications from the electronic prescribing application 12. The healthcare provider device 11, the electronic prescribing application 12, the messaging provider application 14, and the pharmacy device 19 electronically communicate with each other over a network 18, such as the Internet.

In an example embodiment, the electronic prescribing application 12 is used by healthcare providers to prescribe medications to a patient. To do so, the electronic prescribing application 12 enables the healthcare provider to access patient records, search for medications, prescribe medications to patients and send prescriptions to the pharmacy device 19. The electronic prescribing application 12 is hosted (e.g., by a cloud computing service) on a first server 13 that is connected to the network 18. The first server 13 includes a communication module for communicating with the network 18. It is readily understood that the electronic prescribing application 12 may be a standalone application or integrated into an electronic health records (EHR) system. In other embodiments, the electronic prescribing application 12 is replaced with another different type of healthcare interface which enables the healthcare provider to capture input pertaining to a particular encounter with a patient.

In the example embodiment, healthcare providers access the electronic prescribing application 12 by using the healthcare provider device 11. The healthcare provider device 11 may take the form of a desktop computer located in the healthcare provider's office. In other examples, the healthcare provider device 11 may take the form of a mobile computing device, such as an electronic tablet or a smart phone, a laptop computer or another type of computing device. The healthcare provider device 11 is connected to the network 18 and accesses the electronic prescribing application 12 as well as other external resources via the network 18. Examples of external resources that the healthcare provider device 11 may access include but are not limited to websites of medication manufacturers, websites of healthcare insurance providers, other patient care management tools, etc. In one example, the healthcare provider device 11 is connected directly to the network 18. The healthcare provider device 11 may be connect by a wired connection such as Ethernet, or through a wireless communication link, such as the cellular network, Wireless Local Area Network (WLAN), Bluetooth or the like. In another example, the healthcare provider device 11 is connected to the network 18 through a private network, for example the private network in the physician's office.

Access to the electronic prescribing application 12 may be restricted and the healthcare provider device 11 may prompt the healthcare provider to provide login credentials to access the electronic prescribing application 12. In an example embodiment, the healthcare provider device 11 accesses the electronic prescribing application 12 through a web-based user interface over the network 18. The healthcare provider is preferably a physician but can also include a nurse, an assistant to the physician, a medical resident, a medical student, or the like. A healthcare provider who is an authorized user is provided with an authorization token. The authorized user includes the authorization token in requests that are made from the electronic prescribing application 12 to the messaging provider application 14 on behalf of the authorized user. Upon receiving a request with an authorization token embedded in the request, the messaging provider application 14 authenticates the authorization token by verifying the authorization token's authenticity and grants the request if the authorization token is an authentic token.

The messaging provider application 14 is designed to receive input from a healthcare provider and predict an aspect of the therapy for the patient based in part on the input, where the input pertains to a particular encounter of the patient with the healthcare provider. Message content for the healthcare provider is then determined using the predicted aspect of the therapy. Message content may include but is not limited to educational information regarding the disease, diagnosis, therapy, or diagnostic results which are part of the patient's therapy, or promotional information, such as coupons or discounts for medications or therapy. More specifically, the educational information may be regarding possible diseases and diagnoses a patient may have, regarding how to diagnose a disease a symptom set, regarding major diagnostic results to check in a disease or symptom set, regarding meaning of diagnostic elements in a disease or symptom set, regarding biology and chemistry behind a disease or symptom set, regarding therapies which may be indicated or helpful in treating a disease or symptom set, regarding the utility of a therapy for a disease or symptom set, regarding the biology and chemistry of a therapy, and regarding the financial assistance possibilities for a patient who may take the therapy. The techniques described herein may be used to deliver other types of information as well, such as product information, disease information and patient educational information. Lastly, the messaging provider application 14 delivers message content to the healthcare provider during or near the encounter with a patient.

In one example, the electronic prescribing application 12 generates a prescription and sends the prescription electronically to the pharmacy device 19. The electronic prescribing application 12 includes a promotional offer with the prescription that is sent to the pharmacy device 19. The pharmacy device 19, located at a pharmacy, receives electronic prescriptions from the electronic prescribing application 12. In another example, the electronic prescribing application 12 generates a prescription and sends the prescription electronically to the patient. The patient in turn sends the prescription electronically to the pharmacy or brings the prescription to the pharmacy.

The system 10 may also include a pharmaceutical application 16. The pharmaceutical application 16 is hosted (e.g., by a cloud computing service) on a third server 17. The pharmaceutical application 16 is used to define criteria for message content being delivered to the healthcare provider. The message content preferably pertains to a service or product offered by a pharmaceutical company. The message content delivery criteria is accessible to or made available to the messaging provider. For example, the messaging provider application 14 periodically retrieves message content delivery criteria from the pharmaceutical application 16. The messaging provider in turn uses the message content delivery criteria to determine what and when content is to be delivered to the healthcare provider as further described below. In other embodiments, the pharmaceutical company is merely an example of the types of healthcare organizations (e.g., insurance companies) who are interested in delivering messages to a healthcare providers during or near an encounter with a patient.

In an example embodiment, the message provider application 14 is a service provided by a third party independent from the healthcare provider and the pharmaceutical companies. Thus, the messaging provider application 14 is hosted (e.g., by a cloud computing service) on a second server 15 and is accessible to the electronic prescribing application 12 over the network 18. In other embodiments, the message provider application 14 is a service provided by a pharmaceutical company or another type of healthcare organization.

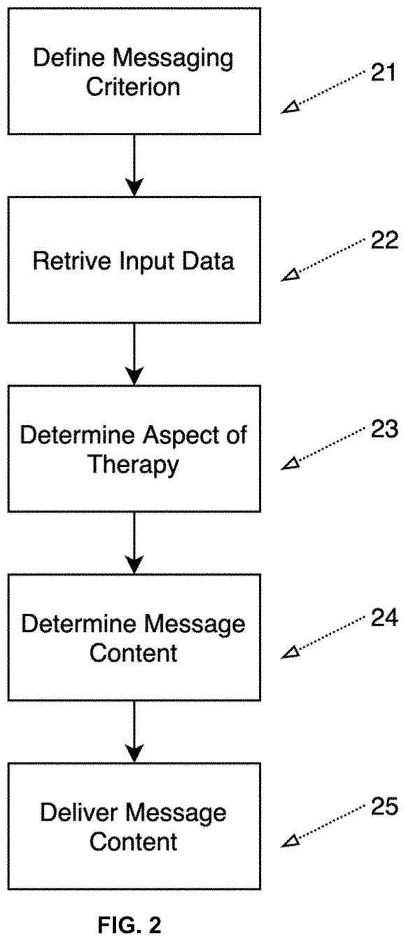

FIG. 2 provides an overview of an improved technique for delivering point or care message content to a healthcare provider. Message content as well the criteria for delivering the message content to a healthcare provider are preferably defined at 21 by a pharmaceutical company or another type of healthcare organization. Although not limited thereto, message content pertains to a service or product offered, for example by the pharmaceutical company. For example, the message content may pertain to a particular medication used to treat a disease and the criteria for the message content is further defined as eligibility rules for a candidate to participate in a clinical trial of the particular medication.

During an encounter with a particular patient, the electronic system associated with healthcare provider captures some information which pertains to the particular patient and physicians involved in the encounter. For example, the healthcare provider may assign a diagnosis code in their Electronic Health Record system which pertains to the patient or associate lab results with the patient, instantiating this data in the electronic system. This information which pertains to the particular encounter is provided to the messaging provider in real-time (i.e., immediately or during the particular encounter with the patient). That is, the information is received by the messaging provider application as input at 22 in real-time from the electronic system used by healthcare provider, where the input pertains to the particular encounter of a given patient with the healthcare provider.

In one example, the input from the patient encounter includes a timestamp for the particular encounter, and at least one of a diagnosis for the given patient or medication prescribed to the given patient and whether the given patient is eligible for a clinical trial is predicted. In another example, the input from the patient encounter includes a timestamp for the particular encounter, identifying information for the healthcare provider, and medication prescribed to the given patient and a diagnostic test for the given patient is predicted. In yet another example, the input from the patient encounter includes identifying information for the healthcare provider, and at least one of medication prescribed to the given patient or medical procedures administered to the given patient, such that a diagnostic test for the given patient and/or a diagnosis for the given patient is predicted. It is readily understood that these examples are merely illustrative and non-limiting.

In many instances, the messaging provider has access to only partial information regarding the patient and their health condition. Data analytics can be used to determine applicable message content to provide a healthcare provider during or near to a particular patient encounter. This approach can be significantly improved by using input pertaining to the current patient encounter with the healthcare provider. This disclosure proposes predicting an aspect of a patient's clinical context using predictive algorithms, such as machine learning, as indicated at 23. The predictions are made in part based on the input pertaining to the current patient encounter with the healthcare provider. For example, a determination is made as to an aspect of a therapy or for an indicator of a therapy for the patient. It is noted that the determined aspect of therapy or indicator thereof is absent from the input data.

Particular message content for the healthcare provider is then determined at 24 using the predicted aspect of the patient's clinical context, where the message content typically pertains to a service or product related to the therapy. The message content is also determined using criteria for delivering the message content as defined, for example by a pharmaceutical company.

Lastly, the particular message content is delivered at 25 to the healthcare provider during or near the encounter with the patient. It is to be understood that only the relevant steps of the technique are discussed in relation to FIG. 2, but that other software-implemented instructions may be needed to control and manage the overall operation of the system.

An example scenario for delivering point of care message content to a healthcare provider is further described. In this scenario, a pharmaceutical company is interested in delivering messages regarding the utility of a particular therapy, such as a medication, to physicians who are treating patients with a diagnosis of the condition of heart failure. The criteria for delivering message content regarding a particular therapy may be defined generally or as a specific rule set. An example of general criteria for message content may be as follows: 1) provide the physician with a message about the utility of a particular therapy for treating a heart failure condition if the patient is likely diagnosed with a heart failure condition (e.g., systolic heart failure) and has diagnostic or lab values indicating the particular therapy (e.g., low heart ejection fraction); 2) provide the physician an educational message about the importance of managing a heart failure condition if the patient is likely diagnosed with a heart failure condition but the patient's diagnostic or lab values do not support the particular therapy; or, otherwise 3) provide the physician a generic message about the pharmaceutical company. This example is merely intended to be illustrative of the general criteria which can be defined for message content. Additionally, the pharmaceutical company may define the message content for each of these three message types.

Alternatively, the pharmaceutical company may define a specific rule set for delivering message content. An example of a specific rule set is set forth as follows: 1) provide the physician with a message about the utility of a particular therapy for treating a heart failure condition if the likelihood that the patient is diagnosed with a heart failure condition exceeds a predefined diagnosis threshold (e.g., greater than 75% likelihood) and the likelihood that the patient has diagnostic or lab values indicating the particular therapy exceed a predefined testing threshold (e.g., greater than 90% likelihood); 2) provide the physician an educational message about the importance of managing a heart failure condition if the likelihood that the patient has diagnostic or lab values indicating the particular therapy exceed the predefined testing threshold but the likelihood that the patient is diagnosed with a heart failure condition falls below the predefined diagnosis threshold; or, otherwise 3) provide the physician a generic message about the pharmaceutical manufacturer. Again, this example is merely intended to be illustrative of a specific rule set which can be defined for message content and other types of rule sets are contemplated by this disclosure.

During an encounter with a particular patient, the healthcare provider's electronic system captures or otherwise determines information which pertains to the particular encounter with the patient. Information or input data pertaining to the patient encounter may include but is not limited to date and time of the encounter; name of the healthcare provider or other identifying information for the healthcare provider (e.g., national provider identifier number), age of the patient, gender of the patient, location of the patient encounter, information pertaining to therapies for which the patient is actively being treated with, such as medications prescribed to the patient, diagnostic tests administered to the patient, results of the diagnostic tests, or a combination thereof. In one example, the input data is a vector comprised of a timestamp for the particular encounter, identifying information for the healthcare provider, and medication prescribed to the given patient. In another example, the input data is a vector comprised of a timestamp for the particular encounter, age of the patient, gender of the patient and medication prescribed to the given patient. In addition to input data for the current encounter, the input data may further include past medical history, such as from earlier patient encounters with the same or different healthcare provider. In this case, the input data is a two dimensional vector, where a row includes data from a given patient encounter and each row corresponds to a different patient encounter. In any case, this information is passed in real-time by the system which the healthcare provider is interacting with (e.g., electronic prescribing application) to the messaging provider. Although the healthcare provider has likely assigned a diagnosis to the patient, the diagnosis code is not passed to or otherwise made available to the messaging provider in this example.

From the input provided by the healthcare provider, the messaging provider determines an aspect of the patient's therapy (or an indication thereof) using prediction algorithms, such as machine learning. In an example embodiment, the input data serves as input to a neural network and the neural network is designed to predict the likelihood that the patient is diagnosed with a heart failure condition as well as to predict the likelihood that the patient has diagnostic or lab values indicating the particular therapy. Thus, in the example embodiment, the neural network outputs a likelihood percentage that the patient is diagnosed with a heart failure condition and a likelihood percentage that the patient has diagnostic or lab values indicating the particular therapy. While particular reference has been made to a neural network, it is readily understood that other type of machine learning methods also fall within the scope of this disclosure, including but not limited to decision trees, support vector machines, regression analysis and genetic algorithms.

Prediction algorithms are created to predict aspects of a therapy or an indicator thereof which is relevant to the pharma company's messaging goals as described in the messaging criterion. Domain knowledge used to create the prediction algorithms include but are not limited to medical ontologies (which specify diagnosis codes map to the disease of heart failure in this example); clinical decision algorithms (from the American Medical Association) recommending what aspects of a disease must be managed; and medical research best practices from peer reviewed medical research, such as publications in the New England Journal of Medicine, describing the laboratory tests and diagnostic values that suggest a patient will be optimally treated by the pharmaceutical company's therapy. Prediction algorithms are designed to incorporate the pharmaceutical company's messaging criterion. That is, when creating the predictive algorithm, design it to predict information that provides elements that can be used to create a best-guess judgment as to whether any criterion for messaging have been met. Prediction algorithms are also designed to use the anticipated input data and generate the desired output.

In the example embodiment, the predictive algorithm is a neural network as noted above. The neural network is designed and developed, including during data exploration, feature exploration, algorithm exploration, and algorithm selection phases, including cycles thereof, using qualitative, non quantitative logical (e.g. ontologies), and quantitative information derived by clinical guidelines (e.g., American Cardiac Association Guidelines), medical research best practice (e.g., accredited best practices from medical journals) and medical ontology content and logic (e.g., concepts and conditional relationships in ontologies like Snowmed, UMLs, etc.). Neural networks can be trained using supervised or unsupervised training methods. The training data for the neural network includes but is not limited to past medical data, electronic health records, lab records, medical claims, medical prescription data, medical procedure data, patient demographic data, healthcare practitioner and patient demographic data. Other types of training data are also envisioned by this disclosure.

In the example embodiment, the prediction algorithms are part of the messaging provider application and executed in real time in response to receiving the input from the healthcare provider interface application. In one embodiment, the messaging provider has access to the past medical data. In this case, the prediction algorithms are trained in advance by the messaging provider and available for use within the messaging provider application. In other embodiments, a data aggregator possesses or owns the past medical data. In these case, the messaging provider collaborates with the data aggregator to create the prediction algorithms. For example, the messaging provider may specify the requirements for the prediction algorithm, including the criteria for delivering message content from the pharmaceutical company. The data aggregator in turn generates the prediction algorithms and passes them back to the messaging provider for use. In another example, the data aggregator supplies the messaging provider with the past medical data needed to generate the prediction algorithm and the messaging provider generates the prediction algorithms. Other variants of collaboration are envisioned by this disclosure.

Given a prediction about an aspect of the patient's clinical context, a determination can be made for the particular message content to be delivered to the healthcare provider. Continuing with the example above, the neural network outputs a likelihood percentage (or certainty estimate) that the patient is diagnosed with a heart failure condition and a likelihood percentage that the patient has diagnostic or lab values indicating the particular therapy. Rules are created to interpret the output and/or translate the output into decisions about message content. In one example, likelihood percentages are translated directly into message content or a type of message content. For example, if the likelihood that the patient is diagnosed with a heart failure condition exceeds a predefined diagnosis threshold (e.g., greater than 75% likelihood) and the likelihood that the patient has diagnostic or lab values indicating the particular therapy exceed a predefined testing threshold (e.g., greater than 90% likelihood), the physician is provided with a message about the utility of a particular therapy for treating a heart failure condition (i.e., “therapy message”); if the likelihood that the patient has diagnostic or lab values indicating the particular therapy exceed the predefined testing threshold but the likelihood that the patient is diagnosed with a heart failure condition falls below the predefined diagnosis threshold, the physician is provided with an educational message about the importance of managing a heart failure condition (i.e., “clinical importance message”); otherwise, the physician is provided with a generic message about the pharmaceutical manufacturer.

In another example, a first rule set is used to interpret the output and then a second rule set is applied to determine the message content. For example, if the likelihood percentage that the patient has heart failure is about a predefined threshold (e.g., greater than 75%), the patient is assumed to have heart failure. Similarly, if the likelihood percentage that the patient had ejection fraction heart test with a low ejection fraction result is above a predefined threshold (e.g., greater than 90%), then patient is assumed to have had this particular diagnostic test with the noted result. A second set of rules is then applied to determine the type of message content. If the patient is assumed to have heart failure and the patient is assumed to have had an ejection fraction heart test with low ejection fraction result, the physician is provided with a message about the utility of a particular therapy for treating a heart failure condition (i.e., “therapy message”). If the patient is assumed to have heart failure but the patient did not have an ejection fraction heart test with low ejection fraction result, the physician is provided with an educational message about the importance of managing a heart failure condition (i.e., “clinical importance message”). In any other case, the physician is provided with a generic message about the pharmaceutical company. This disclosure envisions other ways of translating output from a predictive algorithms to messaging content.

Lastly, message content is delivered by the messaging provider in real or near time back to the healthcare provider. Depending on the message type, the particular message content can be retrieved, for example from a lookup table. In some instances, applying rules to the output of the prediction algorithms can lead to identification of multiple messages that apply to the patient's clinical context and could be sent to the healthcare provider. In one embodiment, each of the identified messages are delivered to the healthcare provider. In other embodiments, the message types are assigned a priority such that only one message is delivered to the healthcare provide or a subset of messages having the highest priority are delivered to the healthcare provider. In the example embodiment, the message content is sent immediately from the messaging provider application to the healthcare provider interface application. In this way, the message is delivered to the point of care during the encounter with the patient. In other embodiments, the message content is delivered to the healthcare provider interface application at a later time proximate to the encounter (e.g., on same day or within a set period of time such as within two hours).

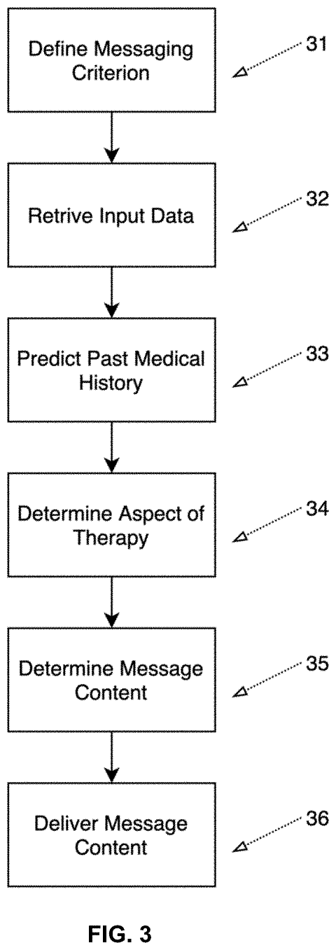

FIG. 3 illustrates another example scenario for delivering point of care message content to a healthcare provider. In this example scenario, a healthcare organization is interested in delivering messages regarding a therapy to treat hypophosphatasia, where the therapy's indication for usage include low bone density and low alkaline phosphatase enzyme levels. Criteria for message content being delivered to the healthcare provider is defined by the healthcare organization as indicated at 21. Example criteria for delivering message content regarding a therapy to treat hypophosphatasia is as follows: 1) provide the physician with a message about the pharmaceutical company's therapy if the patient in an encounter has been given a bone density diagnostic test such as a low-energy X-ray test, the patient in an encounter has had the diagnostic result of low bone density from the bone density diagnostic test and the patient is going to get an Alkaline Phosphatase (ALP) enzyme lab test in the future; 2) provide the physician an education message about hypophosphatasia if the patient in an encounter has been given an bone density diagnostic test such as a low-energy X-ray test, if the patient in an encounter has had the result of low bone density from the bone density diagnostic test but the patient is not going to get an Alkaline Phosphatase (ALP) enzyme lab test; and otherwise 3) provide the physician a message about pharmaceutical company promoting its treatment for metabolic diseases. This example is merely intended to be illustrative of criteria which can be defined for message content. Additionally, the pharmaceutical company defines the particular message content for each of these three message types.

During an encounter with a particular patient, the healthcare provider's electronic system captures or determines information which pertains to the particular encounter with the patient. In this scenario, the information or input data includes an identifier of the healthcare practitioner who is part of the encounter (e.g., in the form of the practitioner's National Provider Identifier (NPI) number), the age of the patient who is part of the encounter, procedures executed for the patient in the encounter (e.g., in the form of the medical codes for the executed procedures), medication prescribed to the patient at the time of the encounter (e.g., in the form of a list of NDC codes for those medications) and the identifier of the facility in which the event occurs (e.g., in the form of the facility's National Provider Identifier (NPI) number). This input data is passed to and received by the message provider as indicated at 32.

In an example embodiment, the input data is formatted in accordance with JSON. For example, the practitioner's NPI number is stored with key “NPI” at the top level of the JSON key-value tree and with the value of the practitioner's NPI as the value. The age of the patient is stored with key “patient_age” at the top level of the JSON key-value tree with the patient's age as the value. Procedures executed for the patient in the encounter are stored with key “procedures” at the top level of the JSON key-value tree and with a list of elements as the value where each element is a procedure executed for the patient in the encounter. Medications prescribed for the patient at the time of the encounter is with key “medications” at the top level of the JSON key-value tree, and with a list of elements as the value where each element is a medication prescribed for the patient at the time of the encounter. Other formats for the input data are contemplated by this disclosure.

In this example scenario, the prediction algorithms are designed to make inferences about a patient's past and/or future medical history. That is, the prediction algorithms determine certainty estimates for a given component of past medical history for the given patient as indicated at 33, where the certainty estimates are based in part on the input data and the given component of past medical history is not present in the input data. Component of past medical history may include but are not limited to a medical procedure for the given patient, a diagnostic test result for the given patient or a laboratory test result for the given patient, a medical diagnosis for the given patient, or a therapy for the given patient. The prediction algorithms may be based on qualitative unstructured knowledge, such as medical and clinical expertise from the most recent medical literature including from the New England Journal of Medicine, the Journal of the American Medical Association, and the Mayo Clinic's disease guidelines for osteoporosis and for metabolic disease, and structured knowledge, such as how medical and clinical codes are used to reflect the qualitative assessments of medical and clinical experts, by using such as the ICD system and the College of American Pathologist's SNOMED classification system. More specifically, the prediction algorithms output certainty measures that certain medical codes appear on the patient's medical records. In this example, the prediction algorithms output certainty measures that the patient in their past medical history (e.g., within the past two years) has a medical code indicating a diagnostic test for bone density, that the patient in their past medical history has a diagnostic test result for a bone density diagnostic test indicating low bone density, and that the patient will have a medical code for a diagnostic test for levels of the ALP enzyme.

In an example embodiment, the prediction algorithm is implemented by support vector machines with supervised learning. Support vector machines can be designed and developed, including during data exploration, feature exploration, algorithm exploration, and algorithm selection phases, including cycles thereof, using qualitative, non quantitative logical (e.g. ontologies), and quantitative information derived by clinical guidelines (e.g., American Cardiac Association Guidelines), medical research best practice (e.g., accredited best practices from medical journals) and medical ontology content and logic (e.g., concepts and conditional relationships in ontologies like Snowmed, UMLs, etc.). Support vector machines can be trained using supervised or unsupervised training methods. The training data for the support vector machines includes but is not limited to past medical data, electronic health records, lab records, medical claims, medical prescription data, medical procedure data, patient demographic data, healthcare practitioner and patient demographic data.

Next, as aspect of therapy (or an indicator thereof) for the patient is determined at 34 using the certainties for medical codes and/or other medical data in the patient's medical history. In one example, rules are defined for translating the certainty estimates into determinations about therapies for the patient. One rule might be if the certain estimate that the patient's past medical history over the past two years includes a code for a bone density test exceeds a predefined threshold (e.g., greater than 95%), conclude that the patient has had a bone density test in the past two years. Another rule might be if the certainty estimate that the patient's past medical history over the past two years includes diagnostic results for a bone density test that shows at least two standard deviations in density below the average exceeds a predefined threshold (e.g., greater than 80%), conclude that the patient has a diagnostic test result of low bone density. Yet another rule might be if the certainty estimate that the patient's future medical history will include a code for the ALP enzyme test in the next year exceeds a predefined threshold (e.g., greater than 90%), conclude the patient will get a diagnostic test for ALP enzyme. In this way, rules can be applied to the certainty estimates output by the prediction algorithms to determine indicators for potential therapies for the patient.

Additional rules are applied to the different indicators for potential therapies to determine the message content as indicated at 35. Continuing with the example scenario, if the patient was deemed to have had a bone density diagnostic test in the past two years, the patient was deemed to have had a diagnostic test result of low bone density in the past two years, and the patient was deemed to have had or will have a diagnostic test for ALP enzyme in the next two years, provide the physician with a message about the pharmaceutical company's therapy for treating hypophosphatasia. If the patient was deemed to have had bone density diagnostic test in the past two years, the patient was deemed to have had a diagnostic test results of low bone density in the past two years but the patient has not or is not likely to get a diagnostic test for ALP enzyme in the next two years, provide the physician an education message about hypophosphatasia. If neither of these two conditions are met, provide the physician a message about the pharmaceutical company promoting its treatment for metabolic diseases. Lastly, the selected message content is delivered by the messaging provider to the healthcare provider.

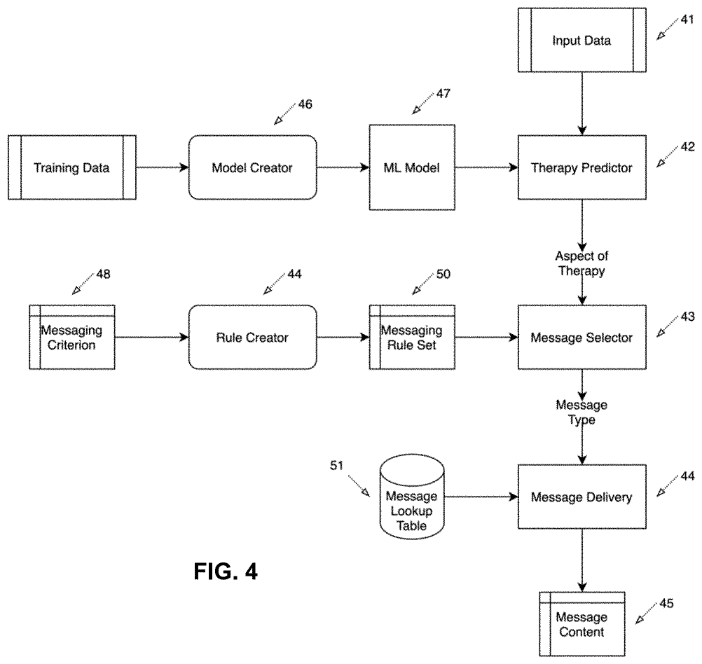

FIG. 4 depicts an example embodiment for the messaging provider application 14. The messaging provider application 14 is comprised generally of a therapy predictor module 42, a message selector module 43 and a message deliverer module 44. The messaging provider application 14 may further include a model creator module 46 and a rule creator module 49. As used herein, the term module may refer to, be part of, or include an Application Specific Integrated Circuit (ASIC), an electronic circuit, a processor that execute one or more software or firmware programs, a combinational logic circuit, and/or other suitable components that provide the described functionality.

The therapy predictor 42 is interfaced with a storage medium 47. The storage medium 47 stores models for one or more predictive machine learning algorithms models. During operation, the therapy predictor 42 receives input data 41 for a given patient from a healthcare provider, for example from an electronic prescribing application. The therapy predictor 42 predicts an aspect of therapy (or an indicator for therapy) for the given patient using predictive machine learning algorithm models. The prediction of the aspect of the patient's therapy is based in part on the input data but the predicted aspect is absent from the input data. As noted above, the input data pertains to a particular encounter between the given patient and the healthcare provider. The model creator 46 receives training data and generates one or more models for use by the predictive machine learning algorithms.

The message selector 43 is interfaced with a messaging rule set 50 and is configured to receive the predicted aspect of the therapy from the therapy predictor 42. The messaging rule set 50 stores criteria for message content to be delivered to a healthcare provider during a patient encounter, where the message criteria is defined, for example by a pharmaceutical company which provides services or products related to potential therapies. The message selector in turn determines particular message content (or type of message content) by applying the message criteria to the predicted aspect of the therapy. In some embodiments, the message criteria may be defined generally as indicated at 48 and a rule creator 49 translates the message criteria 48 into a specific message rule set 50. It is envisioned that this message translation can be performed in an automated way or by a person.

In one embodiment, the message deliverer 44 receive the particular message content from the message selector and deliver the particular message content 45 to the healthcare provider during the particular encounter with the given patient. In other embodiments, the message deliverer 44 receives an indicator of a message type from the message selector. The message deliver 44 uses the message type to retrieve the message content from a lookup table 51 and deliver the message content 45 to the healthcare provider during the particular encounter with the given patient. The message content is preferably delivered to a user interface (e.g., a display) being used by the healthcare provider to access the electronic prescribing application 12. Additionally or alternatively, it is envisioned that the message content may be delivered to another device (e.g., a mobile phone) associated with the healthcare provider and accessible during the patient encounter.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for delivering point of care message content to a healthcare provider, comprising:
defining, by a pharmaceutical company, criteria for message content being delivered to a healthcare provider during a patient encounter, where the message content pertains to a service or product offered by the pharmaceutical company;
receiving, by a messaging provider, input data in real-time from a given healthcare provider, where the input data pertains to a particular encounter of a given patient with the given healthcare provider;
determining an aspect of therapy or an indicator thereof for the given patient using machine learning, where the prediction of the aspect of the therapy or an indicator thereof is based in part on the input data;
determining, by the messaging provider, particular message content for the given healthcare provider using the predicted aspect of therapy or the indicator thereof for the given patient and the criteria for delivering message content, where the particular message content is related to a service or product associated with the therapy of the given patient; and
delivering, by the messaging provider, the particular message content to the given healthcare provider during the particular encounter with the given patient.

2. The method of claim 1 wherein the determined aspect of therapy or indicator thereof is absent from the input data.

3. The method of claim 1 the input includes a timestamp for the particular encounter, and at least one of a diagnosis for the given patient or medication prescribed to the given patient.

4. The method of claim 3 wherein determining an aspect of therapy or indicator thereof for the given patient includes predicting whether the given patient is eligible for a clinical trial, such that the message content pertains to a particular therapy and the criteria for message content being delivered is further defined as eligibility rules for a person to participate in a clinical trial of the particular therapy.

5. The method of claim 1 wherein the input includes a timestamp for the particular encounter, identifying information for the healthcare provider, and medication prescribed to the given patient.

6. The method of claim 5 wherein determining an aspect of therapy or indicator thereof for the given patient includes predicting diagnostic test for the given patient.

7. The method of claim 1 wherein the input includes identifying information for the healthcare provider, and at least one of medication prescribed to the given patient or medical procedures administered to the given patient.

8. The method of claim 7 wherein determining an aspect of therapy or indicator thereof for the given patient includes predicting diagnostic test for the given patient or a diagnosis for the given patient.

9. The method of claim 1 further comprises determining an aspect of therapy for the given patient using a neural network.

10. A computer-implemented method for delivering point of care message content to a healthcare provider, comprising:
defining, by a pharmaceutical company, criteria for message content being delivered to a healthcare provider during a patient encounter, where the message content pertains to a service or product offered by the pharmaceutical company;
receiving, by a messaging provider, input data in real-time from a given healthcare provider, where the input data pertains to a particular encounter of a given patient with the given healthcare provider;
determining certainty estimates for a given component of past medical history for the given patient using supervised learning method, where the certainty estimates are based in part on the input data and the given component of past medical history is not present in the input data;
determining an aspect of therapy or an indicator thereof for the given patient by comparing the certainty estimates for the given component to thresholds;
determining, by the messaging provider, particular message content for the given healthcare provider using the determined aspect of therapy or the indicator thereof for the given patient and the criteria for delivering message content, where the particular message content is related to a service or product associated with the determined therapy of the given patient; and
delivering, by the messaging provider, the particular message content to a device used by the given healthcare provider during the particular encounter with the given patient.

11. The method of claim 10 wherein the input data includes at least one of a therapy for the given patient, a medical procedure for the given patient, a medical diagnosis for the given patient, a diagnostic test result for the given patient or a laboratory test result for the given patient.

12. The method of claim 10 wherein the given component of past medical history for the given patient is one of a therapy for the given patient, a medical procedure for the given patient, a medical diagnosis for the given patient, a diagnostic test result for the given patient or a laboratory test result for the given patient.

13. The method of claim 10 wherein the supervised learning method is further defined as one of multivariate regression, support vector machines, decision trees, or neural networks.

14. The method of claim 10 wherein determining an aspect of therapy or an indicator thereof for the given patient further comprises determining presence of a medical code in an electronic health record for the given patient.

15. The method of claim 14 further comprises determining the presence of the medical code by comparing certainty estimates for the given component of past medical history to thresholds and applying Boolean conditions to the comparison results.

16. A computer-implemented system for delivering point of care message content to a healthcare provider, comprising:
a storage medium for storing predictive machine learning algorithm models;
a therapy predictor interfaced with the storage medium and configured to receive input data for a given patient from a healthcare provider, the therapy predictor operates to predict an indicator of a therapy for the given patient using a predictive machine learning algorithm models, where the input data pertains to a particular encounter between the given patient and the healthcare provider, and the determination of the indicator of therapy is based in part on the input data;
a messaging rule set defines criteria for message content to be delivered to a healthcare provider during a patient encounter, where the message content pertains to therapies for patients;
a message selector interfaced with the messaging rule set and configured to receive the predicted indicator of a therapy for the given patient from the therapy predictor, the message selector operates to determine particular message content for the healthcare provider using the predicted indicator of a therapy and the criteria for message content; and
a message deliverer configured to receive the particular message content from the message selector and deliver the particular message content to the healthcare provider during the particular encounter with the given patient, wherein the therapy predictor, the message selector and the message deliverer are implemented by computer program instructions executed by a computer processor.

17. The system of claim 16 further comprises an electronic prescribing application is implemented by computer program instructions executing on a first server computer, where the electronic prescribing application is configured to capture the input data from the healthcare provider during the particular encounter with the given patient.

18. The system of claim 17 wherein the therapy predictor, the message selector and the message deliverer form a messaging provider application which resides on a second server computer, and the first server computer is connected by a network to the second server computer.

19. The system of claim 17 wherein the input data includes at least one of a medical procedure for the given patient, a medical diagnosis for the given patient, a diagnostic test result for the given patient or a laboratory test result for the given patient, and the predicted indicator of a therapy is absent from the input data.
